(19) United States
(12) Patent Application Publication
(10) Pub. No.: US 2002/0111394 A1
(43) Pub. Date: Aug. 15, 2002

(54) Denture adhesive powder

(75) Inventors: William E. Prosise, Ramsey, NJ (US); Krystyna Plochocka, Scotch Plains, NJ (US)

Correspondence Address: William J. Davis, Esq., INTERNATIONAL SPECIALTY PRODUCTS, Legal Dept., Bldg. 10, 1361 Alps Road, Wayne, NJ, 07470-3529, US

(73) Assignee: ISP INVESTMENTS INC.

(21) Appl. No.: 09/735,562

(22) Filed: Dec. 13, 2000

(51) Int. Cl. C08K 3/00; A61K 6/08

(52) U.S. Cl. 523/116; 523/118

(57) ABSTRACT

This invention relates to a denture adhesive powder composition having extended holding power and markedly increased adhesion properties which comprises an adhesive amount of an inorganic salt of a C₁ to C₄ alkyl vinyl ether/maleic acid polymer wherein 50-90% of the acid moieties of the polymer are in the form of a metal salt and 50-1000 ppm of a mixture containing 30-70 wt. % neutralized or unneutralized ethylenediamine- tetraacetic acid (EDTA) and 70-30 wt. % of an organic, hydroxylated antioxidant and free radical scavenger, said composition in solution with water having a pH above 5.5 and to the method for the preparation of said composition.

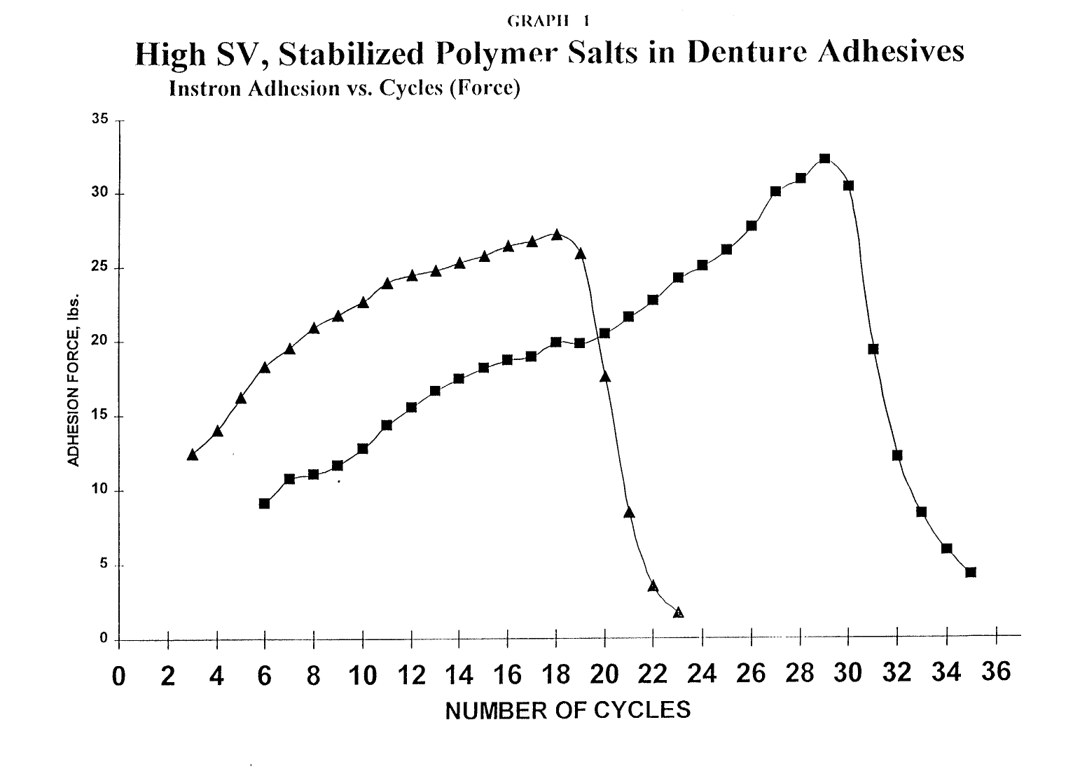

### BACKGROUND OF THE INVENTION

1. Field of the Invention

This invention concerns a denture adhesive powder having markedly increased and extended holding power which comprises a stabilized, salt-neutralized polymer composition containing a co- or ter-polymer of a metal salt of a C₁ to C₄ alkyl vinyl ether monomer and a maleic acid comonomer.

2. Description of the Prior Art

Various adhesive compositions have been employed for affixing dentures or ostomy devices to mucus membranes, several of which involve the use of methyl vinyl ether/maleic anhydride or acid. However, such denture adhesives have not achieved the desired degree of holding power and are effective over a limited period; some over a period of only about 4 hours. Accordingly, it has been the aim of research to extend efficacy and to improve holding strength while avoiding degradation of the polymer as manifested by a loss of molecular weight and viscosity. Among the proposed solutions to one or more of the above problems, Kervinski in U.S. Pat. No. 3,531,427 teaches the polymer mixture with EDTA augmented with an organic EDTA stabilizer selected from the group of (allyl)thiourea, ammonium thiocyanate, tannic acid, vinyl- or polyvinyl-pyrrolidone, pyrogallol, dimethylsulfoxide, diethyldithiocarbamate salt, resorcinol, gallic-, tartaric-, citric-, gluconic- and glycolic-acids. However, it has been found that the EDTA stabilizers were not effective in the stabilization of the polymer unless employed in undesirable large amounts and that many of these stabilizers were toxic even at low concentrations or caused undesirable discoloration of the product. German Patent No. 4,213,972 A1 to Frosch et al. proposes the use of a t-butyl substituted phenol to stabilize the polymer, however, due to the toxicity of phenols in general, this composition has not found acceptance for personal care applications. To avoid the above difficulties, U.S. Pat. Nos. 5,449,715 and 5,739,183 advise stabilizing aqueous solutions of the polymer against molecular weight degradation by addition of a few parts per million (ppm) EDTA salts or superoxide of dismutase. While the later treatments are successful for the intended purposes, they in no way improve the adhesion properties of the composition used in formulations for fixation of dentures.

While the compositions of U.S. Pat. Nos. 5,763,554; 5,395,867 and 5,298,534 utilize salts of methyl vinyl ether/maleic acid copolymer in their formulations to extend adhesive shelf life, they do not increase the adhesive force property of the adhesive formulation. Similarly, the compositions of U.S. Patent Nos. 4,318,742; 5,147,941; 5,959,035 and 6,046,291 have no effect on denture holding power of the powder product.

Accordingly, it is an object of the present invention to provide a denture adhesive powder containing a polymer of an alkyl vinyl ether and an acidic comonomer such as maleic acid or maleic anhydride in a form which not only prevents polymer degradation but actually increases the adhesion force properties of a formulation containing the polymer by at least a 2-fold margin.

It is another object of the invention to provide a solution to the prior problems which does not involve the use of toxic or objectionable color forming additives and which provides an economical and commercially feasible method for increasing holding strength of a denture adhesive.

These and other objects of the invention will become apparent from the following description and disclosure.

### SUMMARY OF THE INVENTION

In accordance with this invention there is provided a denture adhesive composition in the form of a powder comprising a polymer of a C₁ to C₄ alkyl vinyl ether monomer and maleic acid or maleic anhydride comonomer wherein the acidic moieties of the polymer are 50 to 90% neutralized with an inorganic metal oxide and/or carbonate subsequent to the addition of a stabilizing amount of 10-2000 ppm of a mixture of EDTA or a metal salt of EDTA and/or an antioxidant and free radical scavenger selected from a group of organic hydroxy-containing compounds and wherein the powder in solution with water has been adjusted to a pH 5.5 with NaOH, preferably adjusted with NaOH to between about 6 and 8.

### DETAILED DESCRIPTION OF THE INVENTION

The alkyl vinyl ether/maleic acid or anhydride polymers in the composition of the present invention have a number average molecular weight of between about 200,000 and about 5,000,000 and a specific viscosity (1% in MEK at 25° C.) of from about 0.2 to about 15. The present polymers are co- and ter-polymers and optionally contain a minor amount up to 20 wt. % of an additional comonomer such as an α-olefin, particularly isobutylene, a C₈₋₂₀ (meth)acrylate and/or a C₈₋₂₀ alkyl vinyl or allyl ether. Critically, the present polymers of this invention have at least 50% and less than 100% of their acid moieties neutralized in the form of a metal salt by contact with an inorganic metal oxide and/or carbonate. Suitable non-neutralized alkyl vinyl ether/maleic acid polymers are commercially available as, for example GANTREZ® AN series 149, 169 and 179 or S 97 supplied by International Specialty Products.

The C₁ to C₄ alkyl vinyl ether/maleic acid polymers of this invention may also optionally contain a minor amount, e.g. between 0.001 and 5 wt. %, of a crosslinking agent such as a C₄ to C₁₄ diene or another polyfunctional compound.

Suitable inorganic metal neutralizers of this invention are Ca, Na, Mg, Zn, Al, K or Sr-oxides, -carbonates or mixtures of these metals and/or oxides and carbonates, particularly a mixture of Ca and/or Na compounds.

Before drying and grinding to a desired particulate size, the present composition in water is adjusted with NaOH to a pH above 5.5, preferably to a pH of 6-8; CaOH being too insoluble in water to produce the desired affect.

Of the above polymer products, those preferred are the polymers of substantially alternating structure having a mole ratio of between 55:45 and 45:55 C₁ to C₄ alkyl vinyl ether to maleic calcium, sodium salts or mixed calcium/sodium salts, where 60 to 82% of the acid moieties of the polymer are neutralized, and those having Number Average Molecular Weight of from 500,000 to 3,000,000 are most preferred.

The polymer composition of this invention contains 10-2000 ppm of a stabilizer comprising (i) EDTA in an unneutralized, partially neutralized or totally neutralized metal salt form and/or (ii) an organic hydroxylated antioxidant and free radical scavenger or, preferably, a mixture of (i) and (ii). When a mixture is employed, between about 30 to 70 wt. % of the EDTA component and 70 to 30 wt. % of an organic, hydroxylated antioxidant and free radical scavenger is most effective. The function of the EDTA in the present composition is that of a chelating agent for transition metals. Although not preferred, other chelating agents, such as polyphosphates, amine carboxylic acids, such as hydroxy diethylene triamine pentaacetic acid, ethyl ethylene diaminetriacetic acid, nitrile triacetic acid and hydroxy carboxylic acids, such as tartaric acid, citric acid as well as phosphonic acids, such as ethylene diamine tetra(methylene phosphonic acid) can be used to replace, in whole or in part, the EDTA component in the stabilizing EDTA/antioxidant mixture. The neutralized metal salt EDTA in the mixture may contain a metal of a different species from that of the polymer metal salt; however, it is more desirable to employ the same metal as that of the inorganic metal compound employed for reaction with the polymer.

The antioxidant of the mixture is an aliphatic or aromatic hydroxy-containing compound selected from the group of t-butylhydroxyquinone (TBHQ), propyl gallate (PG), N,N-diethylhydroxyamine (DEHA), butylated hydroxyanisole (BHA), butylated hydroxytoluene (BHT), 4-hydroxymethyl-2,6-di-t-butylphenol (HMBP), 2,4,5-trihydroxy butyrophenone (THBP) and mixtures thereof; and is preferably TBHQ and/or PG.

To obtain the composition of this invention, the polymer-chelating agent-antioxidant mixture is contacted with the above inorganic metal compound in an amount sufficient to convert 50-90%, preferably 60-82%, of the —COOH moieties in the mixture, including acid polymer moieties and any unneutralized EDTA, to the corresponding metal salt.

The preparation of the present composition comprises initially contacting a solution of the alkyl vinylether/maleic acid or anhydride polymer in water with the EDTA component and antioxidant to form an aqueous solution containing the unneutralized polymer and 10-2000 ppm, preferably 50-1000 ppm, of the chelating agent and antioxidant mixture. The resulting solution is then reacted, at between about 40° and about 90° C. under agitation, with a water solution of the inorganic metal compound in an amount sufficient to achieve the above critical degree of neutralization of the acid moieties to the corresponding metal salt. Generally, the molequivalent ratio of metal in the reactant inorganic metal compound to polymer acid groups is between about 0.5:1 and about 1:1. Importantly, the desired mixture which results is then adjusted to a pH above 5.5 with NaOH before drying. The neutralized composition is dried at between about 50° and about 90° C. for a period of from about 5 to 40 hours and then ground to a desirable particle size, e.g. an average particle size of from about 5 to about 200μ diameter.

The present powder polymer composition can then be formulated by known methods into a final commercial denture adhesive in the form of a powder, cream or gel base or as a surface coating on the matrix of a denture liner. A suitable viscous formulation comprises from about 10 to about 70 wt. % of the above described polymer product composition, from about 15 to about 30 wt. % mineral and/or vegetable oil and from about 15 to about 30% of a gel oil such as white petrolatum. The formulation can also contain from about 5 to about 30 wt. % of a conventional non-toxic thickener, such as, for example starch, gum arabic, guar gum, gum acacia, agar gum tragacanth, a polyol, or a cellulosic thickener or their mixtures.

Other conventional additives contained in such formulations in known prescribed amounts include food grade coloring and/or flavoring agents, disinfectant, antibiotic and antiseptic agents, sweetener, preservative and the like or mixtures thereof which are further described in U.S. Pat. No. 5,395,867, incorporated herein by reference. Preferably, each of these excipients, when present, are employed at a concentration below 10 wt. % based on the total formulation. The following formulations are representative.

| | Formulation A | Formulation B | Formulation C |
|---|---|---|---|
| | Wt. % | Wt. % | Wt. % |
| 90% Ca/Na neutralized | 33 | 30 | 25 |
| 50:50 MVE/MA | | | |
| polymer (GANTREZ | | | |
| AN 169 ) + 100 ppm | | | |
| TBHQ & EDTA | | | |
| (50% neutralized as | | | |
| Ca/Na salt) | | | |
| White petrolatum | 20 | 10 | 25 |
| Vegetable oil | — | 10 | — |
| Cellulose Thickener* | 27 | 30 | 25 |
| Light mineral oil | 20 | 20 | 25 |

*Na carboxy methyl cellulose

Having generally described the invention, reference is now had to the following examples which illustrate preferred embodiments and provide comparisons with denture adhesives of the prior art. These examples are not to be construed as limiting to the scope of the invention as more broadly defined in the appended claims.

### EXAMPLE 1

| Component | Wt. % |
|---|---|
| Ca/Na polymer** + 100 ppm TBHQ and EDTA | 33 |
| Specific viscosity = about 3 | |
| CMC 7H3SXF (Aqualon)* | 27 |
| White petrolatum | 20 |
| Light mineral oil | 20 |
| Color/Flavor | qs |

*Na carboxy methyl cellulose
**Methyl vinyl ether/maleic acid (1:1)

In the above formula 0.256 equivalents of —COOH groups in the polymer were reacted with 0.178 equivalents of Ca²⁺ and 0.0254 equivalents of Na⁺ to provide 69.5% neutralization of the —COOH polymer groups with Ca and 9.9% of the —COOH polymer groups with Na.

Into a 1-liter jacketed glass kettle equipped with a circulating pump, anchor agitator and reflux condenser, was charged 640 g of deionized water, 0.06 g. of EDTA sodium salt, 0.02 g of TBHQ and 20 g of GANTREZ AN 169 powder (specific viscosity at 1% in MEK at 25° C. was 2.97). The mixture was heated to 85° C. under constant agitation. Upon turning to a slightly hazy viscous solution of hydrolyzed polymer within a period of 1.5 hours, the temperature was reduced to 75° C. and a blend of 8.9 g of CaCO₃ and 2.7 g of Na₂CO₃ was gradually added over a period of 4 hours. After completing the addition, the reaction was maintained for an additional 2 hours at 75° C. The resulting solution of Ca/Na salt of GANTREZ was then adjusted to pH 6.5 with NaOH and subsequently poured onto a glass tray lined with polypropylene film and dried in a forced air oven until a friable, glassy solid was obtained. The dried product was ground to particles of about 100μ average diameter and then redried for an additional 24 hours.

B. Preparation of Formulation #2 (Comparative)

Formulation 2

| Component | Wt. % |
|---|---|
| Ca/Na polymer** having | 33 |
| a specific viscosity of 3.5 | |
| CMC 7H3SXF (Aqualon) | 27 |
| White petroleum | 20 |
| Light mineral oil | 20 |
| Color/Flavor | qs |

The polymer of Formulation 2, having a pH of about 5, was prepared by the method described for formulation 1 in Part A except that the addition of EDTA and TBHQ were omitted and the reaction temperature employed was maintained at 90° C.

### EXAMPLE 2

### Performance Evaluation

In-vitro performance evaluation of the finished denture adhesive is done with the Instron Universal Testing Machine. The denture adhesive is placed between two denture-plastic parallel plates and submerged under artificial saliva. One plate is fixed. The other plate is cycled between 10 pounds compression and complete separation for about 100 cycles. The adhesion, or tension, for each cycle is plotted versus the number of cycles. Three complete runs are averaged for each finished curve.

The formulation and evaluations of two Ca/Na salts of P(MVE/MA) is detailed in graph 1 and summarized in Table 1. In the graph, the performance of formulation 1 is indicated by the symbol ▪ while that of formulation 2 is indicated by the symbol ▴.

TABLE 1

| | Hold Length @ | Max. Hold |
|---|---|---|
| | Maximum, | Strength, |
| Formulation | cycle₁ | lbs.₂ |
| #1 | 29 | 32.2 |
| #2 | 18 | 27.2 |

Notes:
₁Hold length at maximum strength (AKA length of hold) is the cycle number at which maximum hold occurs. Beyond this point, hold decreases rapidly.
₂Maximum hold strength (AKA strength of hold) is the maximum pounds adhesion force reached (Y-axis) by each averaged curve.

What is claimed is:

1. A denture adhesive powder composition having improved adhesion properties which comprises:
(a) an adhesive amount of a polymer of C₁ to C₄ alkyl vinyl ether and maleic acid or anhydride monomers in substantially alternating monomer structure and having 50 to 90% of the acid moieties in the polymer converted to a metal salt and
(b) between about 10 and about 2000 ppm of (i) unneutralized or at least partially neutralized EDTA chelating agent and/or (ii) an organic hydroxylated antioxidant and free radical scavenger, said compostiion having a pH in water adjusted with NaOH to above 5.5.

2. The composition of claim 1 wherein (b) is a mixture of 30-70 wt. % (i) and 70-30 wt. % (ii).

3. The composition of claim 1 having 40-55% of (i) and 55-40% (ii).

4. The composition of claim 1 having 60 to 82% of acid moieties converted to metal salt.

5. The composition of claim 4 wherein metal of the polymer salt is selected from the group of Ca, Na, Mg, Al, K, Zn and Sr.

6. The composition of claim 5 wherein metal of the polymer salt is Ca or Na or a mixture thereof.

7. The composition of claim 5 wherein metal of the polymer salt is mixture of from about 60-70% Ca and from about 8-11% Na.

8. The composition of claim 1 wherein the pH of the composition in water is 6-8.

9. The process for preparing the composition of claim 1 which comprises:
(a) contacting a water solution of a C₁ to C₄ alkyl vinyl ether/maleic acid or anhydride polymer with 10 to 2,000 ppm of (i) EDTA in an unneutralized, partially neutralized or totally neutralized metal salt form and/or (ii) an organic hydroxylated antioxidant;
(b) reacting the solution of (a) with an inorganic metal oxide and/or carbonate employed in an amount sufficient to convert 50-90% of the acid moieties in (a) to the corresponding metal salt;
(c) adjusting the pH of the solution of (b) to above 5.5 with NaOH and
(d) drying and pulverizing the solution of (c) to obtain the powder product of the process.

10. The process of claim 9 wherein said polymer is contacted with a mixture comprising between about 39 and about 70 wt. % (i) and between about 70 and about 30 wt. % (ii).

11. The process of claim 9 wherein the product is pulverized to an average particle size of between about 5 and about 200μ diameter.

12. The process of claim 9 wherein the mixture of (a) is reacted with the inorganic metal compound at a temperature of between about 40° and about 90° C.

13. The process of claim 9 wherein the polymer of (a) has a number average molecular weight of from 200,000 to 5,000,000 and a specific viscosity of from 0.2 to 15.

14. The process of claim 9 wherein the polymer optionally contains up to 20 wt. % of an additional monomer unit selected from the group consisting of an α-olefin, a C₈ to C₂₀ (meth)acrylate, a C₈ to C₂₀ alkyl vinyl or allyl ether and mixtures thereof.

15. The process of one of claims 9 or 12 wherein the polymer optionally contains between about 0.001 and about 5 wt. % of a C₄ to C₁₄ diene crosslinking agent.

16. The process of claim 9 wherein the inorganic metal compound is a Ca, Na, Mg, Zn, Al, K or Sr -oxide or -carbonate or a mixture of said compounds.

17. The process of one of claims 9 or 16 wherein the inorganic metal compound reacted with polymer is employed in an amount sufficient to convert between 60 and 82% of the acid moieties in (a) to the corresponding metal salt.

18. The process of claim 16 wherein the inorganic metal compound is a Ca and/or Na carbonate.

19. The process of claim 9 wherein the pH in (c) is adjusted with NaOH to between 6 and 8.
